(19) United States
(12) Patent Application Publication
(10) Pub. No.: US 2022/0202393 A1
(43) Pub. Date: Jun. 30, 2022

(54) ULTRASONIC SCANNING CONTROL DEVICE, METHOD, AND ULTRASONIC IMAGING SYSTEM

(71) Applicant: GE Precision Healthcare LLC, Wauwatosa, WI (US)

(72) Inventors: Xin Wang, Wuxi (CN); Liye Pei, Wuxi (CN); Hongyu Zhao, Wuxi (CN); Zhenhai Lou, Wuxi (CN); Hongfei Cai, Wuxi (CN)

(21) Appl. No.: 17/560,923

(22) Filed: Dec. 23, 2021

(30) Foreign Application Priority Data
Dec. 31, 2020 (CN) 202011626688.6

(51) Int. Cl. A61B 8/00

(52) U.S. Cl. CPC A61B 8/4461; A61B 8/54; A61B 2562/0247; A61B 8/403; A61B 8/406

(57) ABSTRACT

Provided are an ultrasonic scanning control device and method, and an ultrasonic imaging system. According to an embodiment, the method includes: controlling a scanning assembly to apply an initial pressure to a tissue to be scanned of an subject; obtaining first ultrasonic images at different initial pressures; determining a pressure range on the basis of the first ultrasonic images obtained at the different initial pressures; determining, within the pressure range, a pressure applied by the scanning assembly to the tissue to be scanned during ultrasonic diagnostic scanning; and performing ultrasonic diagnostic scanning on the tissue at the determined pressure within the pressure range.

### TECHNICAL FIELD

The present invention relates to the field of ultrasonic imaging, in particular to an ultrasonic scanning control device and method, an ultrasonic imaging system, and a computer-readable storage medium for performing the ultrasonic scanning control method.

### BACKGROUND

An ultrasonic imaging apparatus usually uses a scanning assembly including an ultrasonic transducer to emit an ultrasonic signal and receive an echo signal so as to perform imaging.

Ultrasonic imaging devices have important applications in scanning of many body organs. For example, a full-field breast ultrasonic scanning device may be used to image breast tissue in one or a plurality of planes. During full-field breast ultrasonic scanning, it is usually necessary for a scanning assembly to apply a certain pressure to a tissue to be scanned (e.g., a breast) so as to press the tissue to be scanned and for imaging. Control and adjustment of the pressure described above are important for scanning imaging. In the prior art, a user needs to spend a long time in adjusting the aforementioned pressure according to experience thereof, and some problems are prone to occur due to improper pressure adjustment. An excessively small or large pressure would affect the quality of an ultrasonic image, and a scanned subject may find an excessively large pressure unbearable, and in this case, it is necessary to completely release the pressure and then perform the steps of pressurization and pressure adjustment again. In addition, an excessively large pressure may further pose a hazard to the safety of the scanned subject.

### SUMMARY

Provided in an aspect of the present invention is an ultrasonic scanning control method, comprising: controlling a scanning assembly to apply an initial pressure to a tissue to be scanned of an subject; obtaining first ultrasonic images at different initial pressures; determining a pressure range on the basis of the first ultrasonic images obtained at the different initial pressures; and determining, within the pressure range, a pressure applied by the scanning assembly to the tissue to be scanned during ultrasonic diagnostic scanning.

Provided in another aspect of the present invention is an ultrasonic scanning control device, comprising: a control module, configured to control a scanning assembly to apply an initial pressure to a tissue to be scanned of an subject; a first image obtaining module, configured to obtain first ultrasonic images at different initial pressures; a pressure range determination module, configured to determine a pressure range on the basis of the first ultrasonic images obtained at the different initial pressures; and a pressure determination module, configured to determine, within the pressure range, a pressure applied by the scanning assembly to the tissue to be scanned during ultrasonic diagnostic scanning.

Provided in another aspect of the present invention is a computer-readable storage medium, the computer-readable storage medium comprising a stored computer program, wherein the above method is performed when the computer program is run.

Provided in another aspect of the present invention is an ultrasonic imaging system, comprising: a scanning assembly, configured to perform reference scanning and formal scanning on a tissue to be scanned of an subject so as to respectively obtain a reference image and an ultrasonic diagnostic image; and a controller, the controller being configured to perform the following operations: controlling, in the reference scanning, the scanning assembly to apply a gradually increasing initial pressure to the tissue to be scanned; determining a pressure range on the basis of reference images obtained at different initial pressures; and adjusting the initial pressure within the pressure range on the basis of a pressure adjustment signal sent remotely, and using the same as a pressure applied by the scanning assembly to the tissue to be scanned during the formal scanning.

It should be understood that the brief description above is provided to introduce, in a simplified form, some concepts that will be further described in the Detailed Description. The brief description above is not meant to identify key or essential features of the claimed subject matter. The scope is defined uniquely by the claims that follow the detailed description. Furthermore, the claimed subject matter is not limited to implementations that solve any disadvantages noted above or in any section of the present disclosure.

### BRIEF DESCRIPTION OF THE DRAWINGS

The present invention will be better understood by reading the following description of non-limiting embodiments with reference to the accompanying drawings, where

FIG. 1 shows a perspective view of an ultrasonic imaging device according to some embodiments;

FIG. 2 shows a cross-sectional view of an internal structure of an ultrasonic imaging system according to some embodiments;

FIG. 3 shows a schematic block diagram of various ultrasonic imaging systems according to some embodiments;

FIG. 4 shows a schematic block diagram of a scanning control device according to some embodiments of the present invention;

FIG. 5 shows a flowchart of an ultrasonic scanning control process according to an example of the present invention; and

FIG. 6 shows a flowchart of a scanning control method according to some embodiments of the present invention.

### DETAILED DESCRIPTION

Specific implementations of the present invention will be described in the following. It should be noted that during the specific description of the implementations, it is impossible to describe all features of the actual implementations in detail in this description for the sake of brief description. It should be understood that in the actual implementation of any of the implementations, as in the process of any engineering project or design project, a variety of specific decisions are often made in order to achieve the developer's specific objectives and meet system-related or business-related restrictions, which will vary from one implementation to another. Moreover, it can also be understood that although the efforts made in such development process may be complex and lengthy, for those of ordinary skill in the art related to content disclosed in the present invention, some changes in design, manufacturing, production or the like based on the technical content disclosed in the present disclosure are only conventional technical means, and should not be construed as that the content of the present disclosure is insufficient.

Unless otherwise defined, the technical or scientific terms used in the claims and the description are as they are usually understood by those of ordinary skill in the art to which the present invention pertains. Terms such as “first,” “second,” and similar words used in this specification and claims do not denote any order, quantity, or importance, but are only intended to distinguish different constituents. “One,” “a(n),” and similar terms are not meant to be limiting, but rather denote the presence of at least one. The term “include,” “comprise,” or a similar term is intended to mean that an element or article that appears before “include” or “comprise” encompasses an element or article and equivalent elements that are listed after “include” or “comprise,” and does not exclude other elements or articles. The term “connect,” “connected,” or a similar term is not limited to a physical or mechanical connection, and is not limited to a direct or indirect connection.

Although some embodiments of the present invention are presented in a particular context of human breast ultrasound, it should be understood that the present invention is applicable to ultrasonic scanning of any externally accessible human or animal body part (for example, abdomen, legs, feet, arms, or neck).

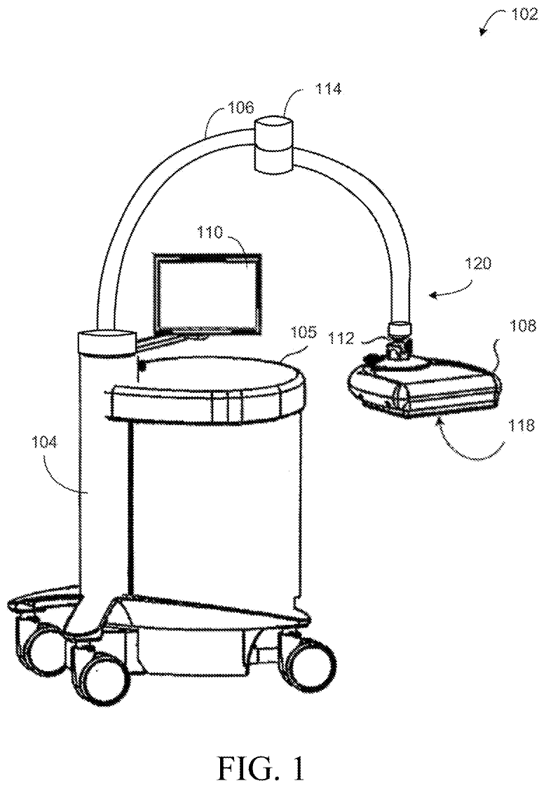

FIG. 1 shows a perspective view of an ultrasonic imaging device 102 according to some embodiments. As shown in FIG. 1, the ultrasonic imaging system 102 includes a frame 104, a processor housing 105, a support arm 106, a scanning assembly 108, and a display 110. The scanning assembly 108 may be connected to a first end 120 of the support arm 106 by means of a ball-and-socket connector (for example, a ball joint) 112. A second end of the support arm 106 is connected to the frame 104 (for example, the second end of the support arm 106 extends into the frame 104).

The display 110 may be connected to the frame 104. In some examples, the display 110 is connected to the frame 104 at a joining point where the support arm 106 enters the frame 104. Since the display 110 is directly connected to the frame 104 rather than the support arm 106, the display 110 does not affect the weight of the support arm 106 and the balancing mechanism thereof.

As described above, the support arm 106 includes a hinge joint 114. The hinge joint 114 divides the support arm 106 into a first arm portion and a second arm portion. The first arm portion is connected to the scanning assembly 108, and the second arm portion is connected to the frame 104. The hinge joint 114 allows the first arm portion to rotate relative to the second arm portion and the frame 104. For example, the hinge joint 114 allows the scanning assembly 108 to translate transversely and horizontally, but not vertically, relative to the second arm portion and the frame 104. In such manner, the scanning assembly 108 may rotate towards the frame 104 or away from the frame 104. However, the hinge joint 114 is configured to allow the entire support arm 106 (for example, the first arm portion and the second arm portion) to move vertically as a whole (for example, translating upwards and downwards as a whole).

In one embodiment, the support arm 106 is configured and adapted so that the scanning assembly 108 is neutrally buoyant in space or has a light net downward weight (for example, 1-2 kg) for pressing the breast, while allowing easy user operation. In an alternative embodiment, the support arm 106 is configured so that a scanning component of the scanning assembly 108 is neutrally buoyant in space when positioned on a tissue to be scanned (for example, a breast tissue) of an subject. Then, after the scanning assembly 108 is in position, internal components of the ultrasonic imaging system 102 may be adjusted to cause the scanning assembly 108 to apply a desired downward weight so as to press the breast and improve image quality. In one example, the downward weight (for example, a force) may be in a range of 2-11 kg.

The scanning assembly 108 may include a housing and a membrane assembly attached to the bottom of the housing. The membrane assembly includes an at least partially fitted membrane 118 in a substantially tensioned state, and the membrane 118 is configured to contact a surface of the tissue when the breast tissue is pressed. A scanner (including, for example, an ultrasonic transducer) of the scanning assembly 108 is provided on an upper surface of the membrane 118 to scan the breast tissue through the membrane 118.

As described above, the scanning assembly 108 is connected to the support arm 106 by means of the ball joint 112. The ball joint 112 may include a locking mechanism for locking the ball joint 112 in place, thereby causing the scanning assembly 108 to remain stationary relative to the support arm 106. Furthermore, the ball joint 112 may also be configured to only rotate but not to move in multiple directions, such as oscillating.

The second end of the support arm 106 may be connected to a load, and the load may increase the pressure and the amount of pressing applied to the tissue on which the scanning assembly 108 is placed. Furthermore, increasing the load applied to the scanning assembly increases the effective weight of the scanning assembly on the tissue to be scanned. In one example, increasing the load may press a tissue of a patient, such as a breast. In such manner, varying amounts of pressure (for example, load) may be applied consistently with the scanning assembly 108 during scanning in order to obtain high-quality images by means of the ultrasonic transducer.

Prior to formal scanning, a user (for example, an ultrasonic technician or a physician) may position the scanning assembly 108 on a patient or a tissue. Once the scanning assembly 108 is correctly fixed in position, the weight (for example, the amount of pressing) of the scanning assembly 108 on the tissue of the patient may be adjusted automatically or manually. Then, the formal scanning process can be started.

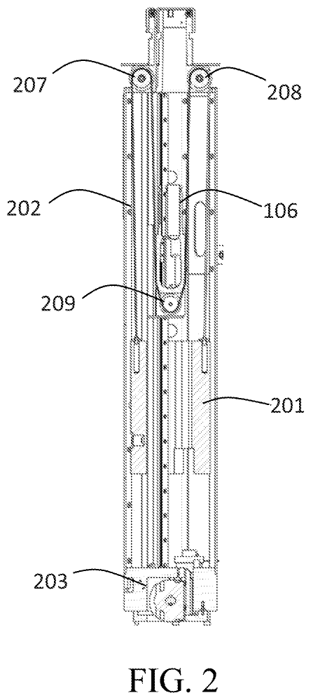

FIG. 2 shows a cross-sectional view of an internal structure of the ultrasonic imaging system 102. Components specifically for effective weight adjustment of the scanning assembly 108 (not shown in FIG. 2) are included in the frame 104 of the ultrasonic imaging system 102. Specifically, a first end of the support arm 106 is connected to the scanning assembly 108 as shown in FIG. 1, and another end of the support arm 106 is disposed in the frame 104. The frame 104 can be used for securing the support arm 106 and guidance during vertical movement. A counterweight 201 is further disposed inside the frame 104. The counterweight 201 may be connected to the second end of the support arm 106 by means of a cable 202. The weight of the counterweight 201 may be approximately equal to the sum of the weight of the scanning assembly 108 and the weight of the support arm 106. In such manner of configuration, the scanning assembly 108 is neutrally buoyant in space, or has a light net upward or downward weight for pressing the breast, while allowing easy user operation. In order to facilitate a sliding connection between the counterweight 201 and the support arm 106, a pulley structure may be provided in an appropriate position. As shown in FIG. 2, two fixed pulleys, a first fixed pulley 207 and a second fixed pulley 208, may be disposed on top of the frame 104. In addition, a movable pulley 209 may be disposed at the bottom of the support arm 106. The cable 202 runs through the aforementioned three pulley structures, and two ends of the cable 202 may be respectively secured to the counterweights 201. In this case, a smooth connection between the counterweight 201 and the support arm can be achieved. As the user presses the support arm 106 downwards, the support arm 106 moves downwards. In this case, the support arm 106 acts on the cable 202 by means of the movable pulley 209 at the bottom, and an upward pulling force applied by the wire 202 to the counterweight 201 increases such that the counterweight 201 is lifted up. Conversely, as the user lifts up the support arm 106, the support arm 106 moves upwards. In this case, a pressure applied by the movable pulley 209 at the bottom of the support arm 106 to the cable 202 decreases. Correspondingly, the pulling force applied by the cable 202 to the counterweight 201 decreases, causing the counterweight 201 to descend.

In addition, a transmission assembly may further be disposed to act on the counterweight 201, thereby acting on the bottom of the support arm 106 and further adjusting the pressure applied by the scanning assembly 108 to the tissue to be scanned. Referring to FIG. 2, in some embodiments, the transmission assembly may include a drive unit 203 and a transmission unit (not shown in the figure). The drive unit 203 acts on the counterweight 201 by means of the transmission unit so as to adjust the pressure applied by the scanning assembly 108 to the tissue to be scanned. In such configuration, the pressure applied by the scanning assembly 108 can be adjusted by electrically controlling the drive unit 203. For example, the user may manually adjust the position of the scanning assembly 108 so that the scanning assembly is close to the surface of the tissue to be scanned. In this case, the pressure applied by the scanning assembly 108 to the tissue to be scanned is still low. Subsequently, in response to a control signal from a control module (for example, the control unit 350 described below), the drive unit 203 may drive the transmission unit to act on the counterweight 201 so as to automatically adjust the pressure as described above.

From the above description, it can be seen that when the drive unit 203 does not act on the counterweight 201, gravity Gweight of the counterweight 201 substantially all acts on the bottom of the support arm 106, that is, a force Fweight applied by the counterweight 201 to the bottom of the support arm 106 is numerically equal to Gweight. As described above, Gweight may be configured to be substantially equal to the sum of gravity Garm of the support arm 106 and gravity Gscanner of the scanning assembly 108. In this case, Fweight=Garm+Gscanner, so that the scanning assembly 108 substantially does not act on the tissue to be scanned. When a specific pressure needs to be applied to the tissue to be scanned, the drive unit 203 may be controlled to apply a driving force Fmotor to the counterweight 201. In this case, Fweight would be less than Garm+Gscanner. The scanning assembly 108 is subjected to unbalanced forces due to the decrease in Fweight, resulting in a pressure Fscanner pressing downwards the tissue to be scanned. In some embodiments, the pressure applied by the scanning assembly 108 to the tissue to be scanned can be obtained by measuring the driving force applied by the drive unit 203 to the counterweight 201.

In some embodiments, the drive unit 203 may include a motor structure. When controlling the drive unit 203, the user can use a controller module (for example, the scanning controller, the scanning control device 400 or the control module 410, the control unit 350, or the ultrasonic engine 318 described below) to send a control signal to the drive unit 203.

In some embodiments, the scanning assembly 108 is configured to move in a direction perpendicular to the tissue to be scanned. In this case, the downward pressure Fscanner of the scanning assembly 108 is numerically equal to the pressure on the tissue to be scanned.

An image processor (not shown in the figure) may further be provided in the processor housing 105 or the scanning assembly 108, and the image processor is configured to generate ultrasonic image data of the breast on the basis of scanning data of the ultrasonic transducer. In some examples, scanning data may be transmitted to another computer system by using any one of a variety of data transmission methods known in the art and for further processing, or the scanning data may be processed by an image processing unit. A general-purpose computer/processor integrated with the image processing unit may further be provided for general user interface and system control. The general-purpose computer may be a self-contained stand-alone unit, or may be remotely controlled, configured, and/or monitored by remote stations connected across networks.

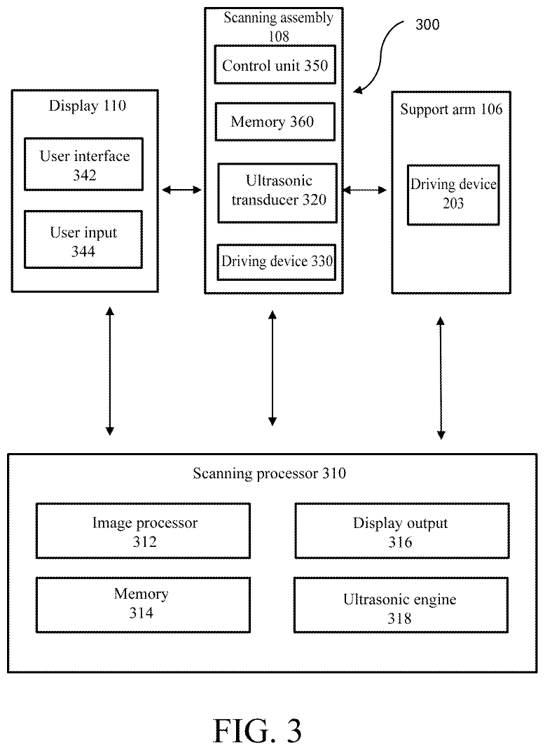

FIG. 3 is a block diagram 300 schematically showing the ultrasonic imaging system 102, the ultrasonic imaging system 102 including the scanning assembly 108, an adjustment arm 106, the display 110, and a scanning processor 310. In one example, the scanning processor 310 may be included in the ultrasonic processor housing 105 of the imaging device 102. As shown in the embodiment of FIG. 3, the scanning assembly 108, the display 110, and the scanning processor 310 are independent components communicating with each other; however, in some embodiments, one or more of these components may be integrated (for example, the display and the scanning processor may be included in a single component).

First, referring to the scanning assembly 108, the scanning assembly 108 includes at least an ultrasonic transducer 320 and a driving device 330. The ultrasonic transducer 320 includes a transducer array of transducer elements, such as a piezoelectric element converting electrical energy into ultrasonic waves and then detecting reflected ultrasonic waves. The ultrasonic transducer 320 and the driving device 330 can specifically be accommodated in a housing of the scanning assembly 108. The housing of the scanning assembly 108 is attached to the support arm 106 and remains stationary during the formal scanning, while the ultrasonic transducer assembly can translate relative to the housing during the formal scanning.

In response to a control signal (for example, from a control unit 350), the driving device 330 may drive the ultrasonic transducer 320 to perform translational scanning on the breast tissue along the membrane 118 during scanning. As shown in FIG. 2, the control unit 350 may be disposed in the scanning assembly 108. In other embodiments, the driving device 330 may also be controlled by a control module disposed in the ultrasonic processor housing 105.

The scanning assembly may further include a memory 360. The memory 360 may be a non-transitory memory, and is configured to store various parameters of the transducer 320, such as transducer usage data (e.g., the number of times of scanning performed, the total amount of time spent in scanning, etc.) as well as specification data of the transducer (e.g., the number of elements of the transducer array, array geometry, etc.) and/or identification information of the transducer module 320, such as a serial number of the transducer module. The memory 360 may include movable and/or permanent devices, and may include an optical memory, a semiconductor memory, and/or a magnetic memory, etc. The memory 360 may include a volatile, non-volatile, dynamic, static, read/write, read only, random access, sequential access, and/or annex memory. In an example, the memory 360 may include a RAM. Additionally or alternatively, the memory 360 may include an EEPROM.

The memory 360 may store non-transitory instructions executable by a controller or a processor (such as a control unit 350) so as to perform one or more methods or routines described below. The control unit 350 can be configured to activate and drive the ultrasonic transducer 320, and can also be configured to control the driving device 203 in the aforementioned support arm 106. However, in other embodiments, the aforementioned operations may also be implemented via a signal from the scanning processor 310.

The scanning assembly 108 optionally communicates with the display 110 so as to instruct a user to reposition the scanning assembly as described above or to receive information from the user (via a user input 344).

Now referring to the support arm 106, and the support arm 106 includes a driving device 203. The driving device 203 is configured to adjust, in response to a control signal, the pressure applied by the scanning assembly 108 attached to the support arm 106 to the tissue to be scanned. The control signal may come from the control unit 350 or the scanning processor 310.

Now referring to the scanning processor 310, and the scanning processor 310 includes an image processor 312, a memory 314, a display output 316, and an ultrasonic engine 318. The ultrasonic engine 318 may drive the activation of the transducer elements of the transducer 320, and in some embodiments, the driving devices 203 and 330 may be activated. Furthermore, the ultrasonic engine 318 may receive raw image data (for example, ultrasonic echoes) from the scanning assembly 108. The raw image data may be sent to the image processor 312 and/or a remote processor (for example, via a network) and be processed to form a displayable image of a tissue sample. It should be understood that in some embodiments, the image processor 312 may be included in the ultrasonic engine 318.

The scanning assembly 108 may communicate with the scanning processor 310 to send raw scanning data to the image processor 312. The scanning assembly 108 may optionally communicate with the display 110 so as to instruct a user to reposition the scanning assembly as described above, or to receive information from the user (via user input 244).

Information may be transmitted from the ultrasonic engine 318 and/or the image processor 312 to a user of the ultrasonic imaging system 102 via the display output 316 of the scanning processor 310. In an example, the user of the scanning device may include an ultrasonic technician, a nurse, or a physician such as a radiologist. For example, a processed image of a scanned tissue may be sent to the display 110 via the display output 316. In another example, information related to parameters of the scanning (such as the progress of scanning) may be sent to the display 110 via the display output 316. The display 110 may include a user interface 342 configured to display images or other information to the user. Furthermore, the user interface 342 may be configured to receive input from the user (such as by means of the user input 344) and send the input to the scanning processor 310. In one example, the user input 344 may be a touch screen of the display 110. However, other types of user input mechanisms are also possible, such as a mouse, a keyboard, and the like.

The scanning processor 310 may further include the memory 314. The memory 314 may include movable and/or permanent devices, and may include an optical memory, a semiconductor memory, and/or a magnetic memory, etc. The memory 314 may include a volatile, non-volatile, dynamic, static, read/write, read only, random access, sequential access, and/or annex memory. The memory 314 may store non-transitory instructions executable by a controller or a processor (such as the ultrasonic engine 318 or the image processor 312) so as to perform one or more methods or routines described below. The memory 314 may further store raw image data received from the scanning assembly 108, processed image data received from the image processor 312 or the remote processor, and/or additional information.

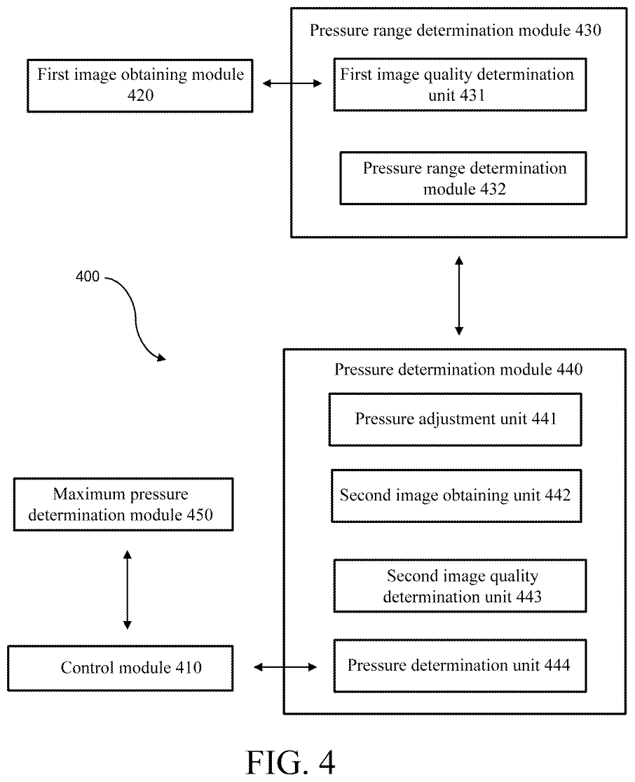

FIG. 4 shows a block diagram 400 of the ultrasonic scanning control device according to one embodiment of the present invention. The ultrasonic scanning control device may communicate with the scanning processor 310 or the control unit 350. The ultrasonic scanning control device may also be integrated in the scanning processor 310 and communicate with other components of the scanning processor 310. At least part of the ultrasonic scanning control device 400 may also be integrated in the ultrasonic engine 318. The ultrasonic scanning control device 400 may also be integrated in the scanning assembly 108, for example, integrated with or communicate with the control unit 350 therein.

As shown in FIG. 4, the ultrasonic scanning control device includes a control module 410, a first image obtaining module 420, a pressure range determination module 430, and a pressure determination module 440.

The control module 410 is configured to control a scanning assembly to apply an initial pressure to a tissue to be scanned of an subject. The structure and principle of the scanning assembly may be similar to those of the aforementioned scanning assembly 108. In one example, when the user places the scanning assembly 108 relatively close to the tissue to be scanned and when the scanning assembly 108 applies a small or minimal pressure to the tissue to be scanned, the control module 410 sends a gradually increasing driving signal to the driving device 203 to cause the driving device 203 to continuously increase a force applied to a load (for example, the aforementioned counterweight 201) of the scanning assembly 108 so as to gradually increase the pressure applied by the scanning assembly 108 to the tissue to be scanned. Since the gradually varied pressure is a pressure adjusted before the formal scanning, the pressure is referred to as an initial pressure.

The first image obtaining module 420 is configured to obtain first ultrasonic images at different initial pressures. In one example, a plurality of first ultrasonic images respectively corresponding to different initial pressures may be obtained in real time during the process in which the initial pressure is continuously varied. At this time, the formal ultrasonic scanning has not been started, and therefore the first ultrasonic image may be different from an ultrasonic diagnostic image obtained during the formal scanning. One of the differences lies in that the first ultrasonic image may be an image generated during a period when the ultrasonic transducer of the scanning assembly 108 is stationary (not driven).

The first image obtaining module 420 may communicate with the aforementioned image processor 312 and therefore may receive the first ultrasonic image from the image processor 312. In other embodiments, the first image obtaining module 420 may include the aforementioned image processor 312.

The pressure range determination module 430 is configured to determine a pressure range on the basis of the first ultrasonic images obtained at the different initial pressures. The pressure range is configured to limit the maximum value and the minimum value of the pressure applied by the scanning assembly 108 to the tissue to be scanned. In addition, determining the pressure range on the basis of corresponding images can prevent subsequent pressure adjustments from exceeding a range required for an image.

In addition, limiting the pressure adjustment to a relatively small range facilitates the process in which a final pressure is determined within this range quickly so that the final pressure is used in the formal scanning. For example, the pressure determination module 440 is configured to determine, within the pressure range, a pressure applied by the scanning assembly to the tissue to be scanned during ultrasonic diagnostic scanning.

The pressure determination module 440 may, for example, communicate with the control module 410 so as to send a determined pressure value, and the control module 410 may then immediately adjust, during the formal scanning process and on the basis of the determined pressure value, the pressure applied by the scanning assembly 108 to the tissue to be scanned.

Optionally, the pressure range determination module 430 may determine a pressure range on the basis of image quality of the first ultrasonic images obtained during the initial pressure adjustment process. As described above, the pressure applied by the scanning assembly 108 to the tissue to be scanned affects the image quality. For example, when the pressure is insufficient, an echo signal received by the ultrasonic transducer may be weak or uneven, and problems such as shadowing and excessive attenuation are prone to occur on the image. In one example, image quality analysis is performed on these first ultrasonic images so as to select first ultrasonic images meeting an image quality requirement therefrom, and initial pressures corresponding to these images meeting the requirement are recorded so as to determine a pressure range.

In one embodiment, the pressure range determination module 430 includes a first image quality determination unit 431 and a pressure range determination unit 432.

The first image quality determination unit 431 is configured to determine, on the basis of a trained deep learning network, whether the first ultrasonic image meets the image quality requirement. The pressure range determination unit 432 is configured to determine the pressure range on the basis of the initial pressures corresponding to the first ultrasonic images meeting the image quality requirement.

In one example, the deep learning network is obtained from training by using a data set of breast ultrasonic images having different qualities as a model input set and using actual quality evaluation results of these inputted images as a model output set. These actual quality evaluation results may be comprehensive results of different indices such as a score, and may also be respectively related to a plurality of quality indices, where these quality indices may include uniformity, resolution, shadowing, an attenuation value, etc.

In such manner, images meeting a quality requirement can be quickly selected from a plurality of first ultrasonic images so as to determine a corresponding pressure range instead of merely observing an image while determining whether to continue to perform pressure adjustment during a pressure adjustment stage, thereby facilitating a subsequent process in which a preferred pressure value for the formal scanning is determined within a limited range, and avoiding problems such as low efficiency and a great error caused by tentative adjustments.

As discussed herein, the deep learning technology (also referred to as deep machine learning, hierarchical learning, deep structured learning, or the like) can employ a deep learning network (for example, an artificial neural network) to process input data and identify information of interest. The deep learning network may be implemented using one or a plurality of processing layers (such as an input layer, a normalization layer, a convolutional layer, a pooling layer, and an output layer, where processing layers of different numbers and functions may exist according to different deep learning network models), where the configuration and number of the layers allow the deep learning network to process complex information extraction and modeling tasks. Specific parameters (or referred to as “weight” or “bias”) of the network are usually estimated through a so-called learning process (or training process). The learned or trained parameters usually result in (or output) a network corresponding to layers of different levels, so that extraction or simulation of different aspects of initial data or the output of a previous layer usually may represent the hierarchical structure or concatenation of layers. During image processing or reconstruction, this may be represented as different layers with respect to different feature levels in the data. Thus, processing may be performed layer by layer. That is, “simple” features may be extracted from input data for an earlier or higher-level layer, and then these simple features are combined into a layer exhibiting features of higher complexity. In practice, each layer (or more specifically, each “neuron” in each layer) may process input data as output data for representation using one or a plurality of linear and/or non-linear transformations (so-called activation functions). The number of the plurality of “neurons” may be constant among the plurality of layers or may vary from layer to layer.

As discussed herein, as part of initial training of a deep learning process used to solve a specific problem, a training data set includes a known input value (for example, a breast ultrasonic image having a known image quality evaluation) and an expected (target) output value (for example, the known quality evaluation result) finally outputted in the deep learning process. In this manner, a deep learning algorithm can process the training data set (in a supervised or guided manner or an unsupervised or unguided manner) until a mathematical relationship between a known input and an expected output is identified and/or a mathematical relationship between the input and output of each layer is identified and represented. In the learning process, (part of) input data is usually used, and a network output is created for the input data. Afterwards, the created network output is compared with the expected output of the data set, and then a difference between the created and expected outputs is used to iteratively update network parameters (weight and/or bias). A stochastic gradient descent (SGD) method may usually be used to update network parameters. However, those skilled in the art should understand that other methods known in the art may also be used to update network parameters. Similarly, a separate validation data set may be used to validate a trained learning network, where both a known input and an expected output are known. The known input is provided to the trained learning network so that a network output can be obtained, and then the network output is compared with the (known) expected output to validate prior training and/or prevent excessive training.

The pressure determination module 440 may specifically include a pressure adjustment unit 441, a second image obtaining unit 442, a second image quality determination unit 443, and a pressure determination unit 444.

The pressure adjustment unit 441 is configured to receive a pressure adjustment signal based on an operation of the aforementioned subject to be scanned (for example, a patient) so as to adjust the current initial pressure. In addition, the pressure adjustment unit 441 is further configured to communicate with the control module 410 so as to send the initial pressure value adjusted by the subject to be scanned, so that the control module 410 adjusts the pressure applied by the scanning assembly 108 to the tissue to be scanned to the adjusted initial pressure value.

In one example, the pressure adjustment unit 441 receives, by means of a remote communication interface (not shown in the figure), the pressure adjustment signal sent by the subject to be scanned, and sends the same to the control module 410. The remote communication interface may be provided in, for example, the scanning assembly 108.

In one example, for example, if the initial pressure value has been adjusted by the control module 410 to a relatively high value, and in this case if the subject to be scanned feels a strong sense of discomfort, then the subject can send a pressure adjustment signal by themselves to reduce the pressure to an acceptable value. As described above, in order to take the image quality into account, pressure adjustment performed by the subject is limited to the aforementioned pressure range determined on the basis of the first ultrasonic images.

In one example, the subject to be scanned may send the pressure adjustment signal by means of a remote control communicating with the remote communication interface or by means of an operation button provided on a hospital bed. Specifically, the remote control or the operation button may include a portion configured to control the pressure to increase and a portion configured to control the pressure to decrease, and may further include an emergency stop control portion as will be described below.

The second image obtaining unit 442 is configured to obtain a second ultrasonic image at the adjusted pressure. In one example, the control module 410 may further activate the ultrasonic transducer again when receiving the pressure adjustment signal so as to cause the ultrasonic transducer to generate scanning data after pressure adjustment. The second image obtaining module 442 may process the scanning data to generate a second ultrasonic image. The second ultrasonic image may also be generated by the aforementioned image processor 312 and sent to the second image obtaining module 442. At this time, the formal ultrasonic scanning has still not been started, and therefore the second ultrasonic image may be different from the ultrasonic diagnostic image obtained during the formal scanning. One of the differences lies in that the second ultrasonic image may be an image generated during a period when the ultrasonic transducer of the scanning assembly 108 is stationary (not driven).

The second ultrasonic image may be used to further determine whether the pressure adjusted by the subject meets the image quality requirement. On this basis, a second image quality determination unit 443 is provided and is used to determine whether the second ultrasonic image meets the image quality requirement.

If the second ultrasonic image meets the image quality requirement, then the pressure determination unit 444 may determine the adjusted pressure as a pressure applied by the scanning assembly 108 to the tissue to be scanned during the ultrasonic diagnostic scanning, and the pressure may be sent to the control module 410.

In one example, if the second ultrasonic image does not meet the image quality requirement, then the subject to be scanned may continue to perform the pressure adjustment operation until an image meeting the image quality requirement is generated. For example, if the adjusted pressure does not meet the image quality, then the second image quality determination unit 443 may send an indication signal via, for example, the user interface 342 of the display 110 so as to notify the user and the subject to be scanned that this adjustment does not meet the image quality requirement. The indication signal may also be directly provided to the subject to be scanned via an audio device, an optical device, etc.

Similar to the first image quality determination unit 431, the second image quality determination unit 443 is configured to determine, on the basis of a trained deep learning network, whether the second ultrasonic image meets the image quality requirement. In one embodiment, the first image quality determination unit 431 may also be used as the second image quality determination unit 443.

Further, the aforementioned indication signal may also be generated when the adjusted pressure reaches the maximum value or the minimum value of the pressure range. For example, if the adjusted pressure reaches the minimum value of the pressure range, then it means that reducing the pressure further would affect the image quality. Therefore, the control module 410 may control the scanning assembly to stop reducing the pressure on the tissue to be scanned, and at the same time send an indication signal to instruct the subject to stop reducing the pressure further. As another example, if the adjusted pressure reaches the maximum value of the pressure range, then it means that the pressure limit is reached. In this case, the control module 410 may control the scanning assembly to stop increasing the pressure on the tissue to be scanned, and at the same time send an indication signal to instruct the subject to stop increasing the pressure further.

In other embodiments, if the pressure adjustment unit 441 does not receive any pressure adjustment signal based on an operation of the subject before the formal scanning is started, then it means that all initial pressures within the pressure range that have been applied during this process are acceptable. In this case, the pressure determination unit 441 (which may also be controlled on the basis of an operation performed by an operation technician) may determine a relatively large pressure within the pressure range as a pressure for the formal scanning so as to guarantee the image quality of the formal scanning to a greater extent.

As described above, the control module 410 is configured to gradually vary the pressure value during initial pressurization performed on the tissue to be scanned. A pressure applied to the tissue to be scanned may exceed an actual acceptable range of the subject if a physician or a technician may focus on the image quality but make a wrong determination on the acceptable range of the subject or fail to stop pressurization in time.

In order to avoid the aforementioned problem, the scanning control device according to the embodiments of the present invention may further include a maximum pressure determination module 450. The maximum pressure determination module 450 is configured to determine, on the basis of inputted subject information, the maximum pressure applied by the scanning assembly to the tissue to be scanned. In addition, when applying the aforementioned gradually varied initial pressure to the tissue to be scanned, the control module 410 ensures that the maximum value of the pressure does not exceed the maximum pressure determined by the maximum pressure determination module 450, that is, the maximum value of the initial pressure is less than or equal to the maximum pressure. Therefore, the control module 410 is configured to obtain the maximum pressure before applying the aforementioned initial pressure to the tissue to be scanned.

In one example, the control module 410 may be configured to receive the pressure applied by the scanning assembly to the tissue to be scanned sent via a pressure sensor (which may be provided on, for example, the scanning assembly) and control, when the sensed pressure value exceeds the aforementioned maximum pressure, the scanning assembly to release the pressure from the tissue to be scanned.

The aforementioned pressure sensor may be disposed between the ultrasonic transducer 320 and the ball joint 112.

In one example, the maximum pressure determination module 450 may receive basic information of the subject to be scanned inputted via the user interface 342 of the display 110 and obtain, by means of analysis and on the basis of the basic information, the maximum pressure that is acceptable to be applied to a part to be scanned of the subject, namely, the maximum pressure applied by the scanning assembly to the tissue to be scanned. Inputting of the aforementioned basic information is usually completed before scanning preparation.

In one example, the maximum pressure associated with each piece of basic information may be calculated on the basis of a preset algorithm between the basic information and the maximum pressure, then weights may be respectively assigned to the plurality pieces of basic information of the subject, and then a comprehensive maximum pressure evaluation result is calculated on the basis of the weights.

In one example, the plurality pieces of basic information may include age, gender, height, weight, medical history, etc.

In one example, the maximum pressure may be quickly found on the basis of a pre-stored and updated lookup table.

Optionally, the aforementioned control module 410 is further configured to control the scanning assembly to release the initial pressure when the initial pressure applied by the scanning assembly to the tissue to be scanned exceeds the maximum pressure. In this case, gradual pressurization may be performed again from, for example, a relatively small initial pressure or from the minimum initial pressure.

The above describes the embodiments in which the scanning assembly is controlled, before the formal scanning is performed on the tissue to be scanned, to press the tissue to be scanned and the pressing force/pressure is adjusted and determined. The embodiments of the present invention may further include performing formal scanning (ultrasonic diagnostic scanning) on the basis of the determined pressure.

For example, the control module 410 may further perform, on the basis of the pressure determined within the pressure range, ultrasonic diagnostic scanning on the tissue to be scanned. In this case, the control module 410 may communicate with the aforementioned ultrasonic engine 318 or control unit 350. Alternatively, the control module 410 is at least partially integrated in the ultrasonic engine 318 or integrated with the control unit 350 so as to start the formal scanning and ensure that the pressure applied by the scanning assembly to the tissue to be scanned during the formal scanning process is the aforementioned pressure value determined by the pressure determination module 440. Starting the formal scan may specifically include, for example, activating the ultrasonic transducer in the scanning assembly 108 and controlling the same to slide (for example, through the membrane 118) in a translational manner along the surface of the tissue to be scanned, for example, to slide from a first edge of a housing where the ultrasonic transducer is located to a second edge opposite the first edge so as to complete scanning of an entire region of interest.

The control module 410 may further be configured to receive an emergency stop signal based on an operation of the subject to be scanned so as to stop the ultrasonic scanning, and control the ultrasonic transducer to withdraw from a current position so as to avoid a pain point. In one example, if an emergency (such as sudden intolerable pain) occurs during the formal scanning, then the subject to be scanned may send an emergency stop signal to the control module 410 by means of, for example, the aforementioned remote control via the remote communication interface. After receiving the emergency stop signal, the control module 410 may control the ultrasonic transducer to withdraw from a current position, for example, to translate to the first edge in a direction opposite to the direction of movement from the first edge to the second edge, and this may still be achieved by controlling the driving device 330.

As described above, the ultrasonic scanning control device according to the embodiments of the present invention may be disposed in the ultrasonic scanning assembly 108, or may communicate with or be integrated with the scanning processor 310, or may also be at least partially integrated with the ultrasonic engine. In other embodiments, the ultrasonic scanning control device may also be disposed in other components of the ultrasonic imaging system, and may also be a separate device independent of the ultrasonic imaging system.

On the basis of the foregoing description, the embodiments of the present invention may further provide an ultrasonic imaging system, and the ultrasonic imaging system includes a scanning assembly such as the aforementioned component 108 and a controller.

The controller is configured to perform the following operations: controlling, in reference scanning, the scanning assembly to apply a gradually increasing initial pressure to a tissue to be scanned; determining a pressure range on the basis of reference images obtained at different initial pressures; and adjusting the initial pressure within the pressure range on the basis of a pressure adjustment signal sent remotely, and using the same as a pressure applied by the scanning assembly to the tissue to be scanned during the formal scanning.

The aforementioned reference scanning may include, before the formal scanning and during adjustment of the aforementioned pressure, the process in which scanning is performed on the tissue to be scanned and image data is generated. In the reference scanning, the scanning assembly 108 can move, in a vertical direction, towards or away from the tissue to be scanned so as to increase or reduce the pressure applied thereby to the tissue to be scanned, and the ultrasonic transducer in the scanning assembly is stationary relative to the housing thereof.

The aforementioned formal scanning is a process configured to perform translational scanning, by means of the ultrasonic transducer sliding in a translational manner, on the tissue to be scanned and generate image data when the housing of the scanning assembly is disposed stationary relative to the tissue to be scanned.

Optionally, the aforementioned controller may also determine, on the basis of the basic information of the subject to be scanned, the maximum pressure that is acceptable to be applied to the tissue to be scanned, and control, in the reference scanning, the initial pressure applied by the scanning assembly to the tissue to be scanned to not exceed the maximum pressure.

Optionally, the ultrasonic imaging system may include a pressure sensor. The pressure sensor is configured to sense the pressure applied by the scanning assembly to the tissue to be scanned and send the same to the controller. The controller is further configured to determine whether the pressure sent by the pressure sensor exceeds the maximum pressure determined on the basis of the basic information of the subject and if so, control the scanning assembly to release the pressure from the tissue to be scanned.

Optionally, the controller may further determine whether a reference image obtained at the adjusted initial pressure meets the image quality requirement, and if so, determine the adjusted initial pressure as the pressure applied by the scanning assembly to the tissue to be scanned during the aforementioned formal scanning.

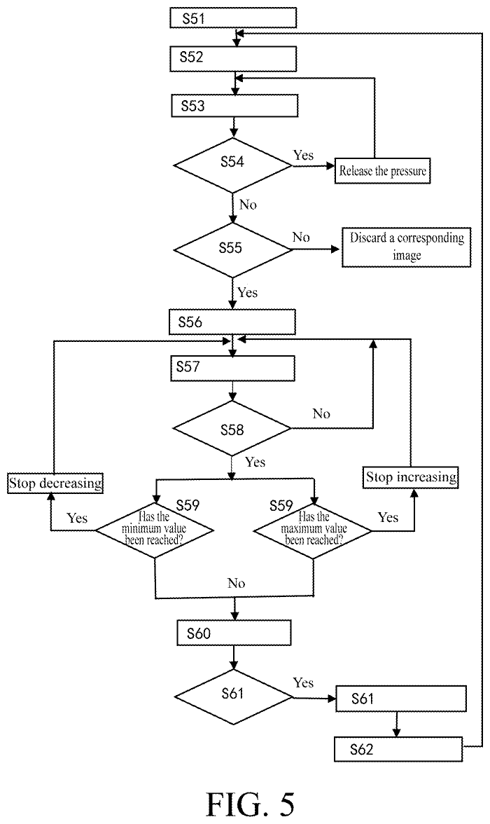

On the basis of the foregoing description, the embodiments of the present invention may provide an example of an ultrasonic scanning control process. A flowchart of this example is shown in FIG. 5.

In step S51, basic information of an subject is received. The basic information is inputted into the ultrasonic imaging system via the user interface of the display 110.

In step S52, the maximum pressure that is acceptable to be applied to a tissue to be scanned of the subject is determined on the basis of the basic information of the subject.

In step 53, a scanning assembly is controlled, in response to a control signal based on an operation of an operator, to start to apply an initial pressure to the tissue to be scanned of the subject. In this step, this operation may include an operation performed on a control button disposed on any component of the ultrasonic imaging system, and may also include an operation of directly pressing the support arm 106. After this operation is triggered, the initial pressure may be gradually increased in relatively small steps via a control module (for example, the module 410).

In step S54, a pressure sensor senses whether a pressure applied by the scanning assembly to the tissue to be scanned exceeds the maximum pressure. If a sensing result is “yes,” then the pressure is released, and the process returns to step S51 so as to re-perform pressure control. If the sensing result is “no,” then step S55 is executed.

Step S55 may be executed generally in synchronization with step S53. In step S55, whether image quality of first ultrasonic images obtained at different initial pressures applied in step S53 meets the requirement is determined on the basis of a deep learning network. If a determination result is “yes,” then an initial pressure corresponding thereto is recorded, and if the determination result is “no,” then this image may be discarded.

In step S56, a pressure range is determined on the basis of the recorded initial pressures.

In step S57, the current initial pressure is finely adjusted on the basis of a pressure adjustment signal from the subject, and then step S58 is executed.

In step S58, whether a second ultrasonic image generated at the finely adjusted pressure meets the requirement is determined on the basis of a deep learning network. If a determination result is “yes,” then step S59 is executed, and if the determination result is “no,” then the process returns to step S57. If a pressure adjustment signal from the subject is not received, then the current unadjusted pressure is considered to be acceptable by the subject, and the process may proceed directly from step S56 to step S58. In addition, in step S58, the unadjusted pressure is used as the aforementioned “finely adjusted pressure,” and whether the same meets the requirement is determined. If a determination result is “yes,” then step S59 is executed, and if the determination result is “no,” then the process returns to step S57.

In step S59, whether the current pressure has reached the maximum value or the minimum value of the aforementioned pressure range is determined. If a determination result is “no,” then step S60 is executed, and if the determination result is “yes,” then a corresponding pressure increasing operation or pressure reducing operation is stopped and the process returns to step S57.

In step S60, formal ultrasonic scanning is performed on the basis of the finely adjusted pressure or a pressure not being subjected to fine adjustment. Before step S60 is executed, steps S57-S59 may be repeatedly executed on the basis of multiple fine adjustment operations of the subject, and then the same operation may be performed again according to a next adjustment signal until the operator determines that a final adjusted pressure can be applied to the formal scanning. For example, if the pressure adjustment signal is received again in any process of steps S57-S59, then the current process may be interrupted, and the fine adjustment operation may be performed again from step S57. For safety considerations, an interruption may not be performed, and instead, S57-S59 are cyclically executed according to adjustment signals received multiple times, until an adjusted pressure is determined. The determination may be achieved by means of, for example, a control button.

In step S61, whether an emergency stop control signal is received is determined, and if a determination result is “yes,” then step S61 is executed.

In step S61, the ultrasonic imaging system is controlled to stop performing the current scanning.

In step S62, the ultrasonic transducer is controlled to withdraw from a current position, and the process returns to step S52.

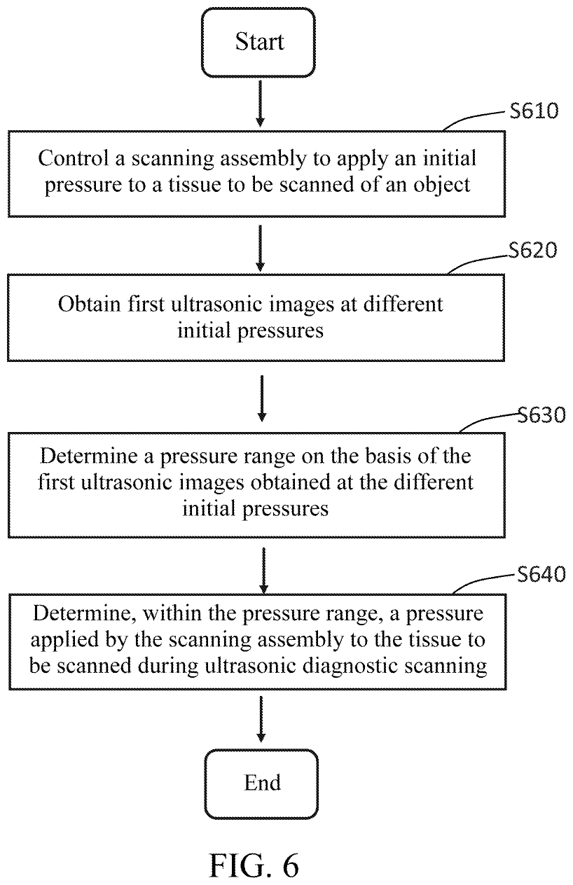

FIG. 6 shows a flowchart of the ultrasonic scanning control method according to one embodiment of the present invention. As shown in FIG. 6, the method includes steps S610, S620, S630, and S640.

In step S610, a scanning assembly is controlled to apply an initial pressure to a tissue to be scanned of an subject. In step S620, first ultrasonic images are obtained at different initial pressures. In step S630, a pressure range is determined on the basis of the first ultrasonic images obtained at the different initial pressures. In step S640, a pressure applied by the scanning assembly to the tissue to be scanned during ultrasonic diagnostic scanning is determined within the pressure range.

Optionally, step S630 includes: determining, on the basis of a trained deep learning network, whether the first ultrasonic image meets an image quality requirement; and determining the pressure range on the basis of initial pressures corresponding to the first ultrasonic images meeting the image quality requirement.

Optionally, step S640 includes: receiving a pressure adjustment signal based on an operation of the subject so as to adjust the current initial pressure; obtaining a second ultrasonic image at the adjusted pressure; and determining whether the second ultrasonic image meets the image quality requirement, and if so, then determining the adjusted pressure as the pressure applied by the scanning assembly to the tissue to be scanned during ultrasonic diagnostic scanning.

Further, whether the second ultrasonic image meets the image quality requirement is determined on the basis of a trained deep learning network.

Optionally, step S640 includes: if the adjusted pressure reaches the minimum value of the pressure range, then controlling the scanning assembly to stop reducing the pressure applied on the tissue to be scanned; and if the adjusted pressure reaches the maximum value of the pressure range, then controlling the scanning assembly to stop increasing the pressure applied on the tissue to be scanned.

In other embodiments, the method may further include the following step: if the second ultrasonic image does not meet the image quality requirement or if the adjusted pressure reaches the maximum value or the minimum value of the pressure range, then issuing a corresponding indication signal.

In other embodiments, before step S610, the method may further include: determining, on the basis of received basic information of an subject, the maximum pressure applied by the scanning assembly to the tissue to be scanned; wherein the maximum value of the initial pressure is less than or equal to the maximum pressure.

In other embodiments, the method may further include the following steps: receiving a feedback from a pressure sensor indicating whether the value of the pressure applied by the scanning assembly to the tissue to be scanned exceeds the maximum pressure, and if a determination result is “yes,” then controlling the scanning assembly to release the initial pressure. If the determination result is “no,” then step S630 is executed.

In other embodiments, the method may further include the following steps: performing, on the basis of the determined pressure within the pressure range, the ultrasonic diagnostic scanning on the tissue to be scanned; and receiving an emergency stop signal based on an operation of the subject so as to stop the ultrasonic diagnostic scanning.

The method may further include the following step: controlling the ultrasonic transducer to withdraw from a current position on the basis of the emergency stop signal.

The embodiments of the present invention may further provide a computer-readable storage medium, and the computer-readable storage medium includes a stored computer program, wherein the method of any one of the above embodiments of the claims is performed when the computer program is run. The computer program may be stored in, for example, the memory 314 or 360.

In the present invention, the pressure range is determined on the basis of the ultrasonic images obtained at the different pressures, so that the finally determined pressure for scanning is within the acceptable range of the image, thereby avoiding image quality problems caused by improper pressure configurations.

In addition, the maximum pressure is determined on the basis of the information of the patient, thereby avoiding the problem of improper pressure configurations resulting from succinct determination, and guaranteeing image quality to the greatest extent.

In addition, the pressure is adjusted within the acceptable pressure range of the image on the basis of the operation of the subject, thereby ensuring the image quality, user safety, friendliness, and the like.

In addition, the deep learning network is used to determine whether image quality at a certain pressure meets the requirement, thereby avoiding the problem of low efficiency caused by the adjusted pressure not meeting the image requirement. In addition, the resulting pressure adjustment range is relatively accurate, thereby avoiding the case in which the pressure is excessively large or the pressure is insufficient.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

1. An ultrasonic scanning control method, comprising:
controlling a scanning assembly to apply an initial pressure to a tissue to be scanned of an subject;
obtaining first ultrasonic images at different initial pressures;
determining a pressure range on the basis of the first ultrasonic images obtained at the different initial pressures;
determining, within the pressure range, a pressure to be applied by the scanning assembly to the tissue to be scanned during ultrasonic diagnostic scanning; and
performing ultrasonic diagnostic scanning on the tissue at the determined pressure within the pressure range.

2. The method according to claim 1, wherein said determining a pressure range on the basis of the first ultrasonic images obtained at the different pressures comprises:
determining, on the basis of a trained deep learning network, whether the first ultrasonic images meet an image quality requirement; and
determining the pressure range on the basis of initial pressures corresponding to the first ultrasonic images meeting the image quality requirement.

3. The method according to claim 1, wherein the step of said determining, within the pressure range, a pressure applied by the scanning assembly to the tissue to be scanned during ultrasonic diagnostic scanning comprises:
receiving a pressure adjustment signal based on an operation of the subject so as to adjust the current initial pressure;
obtaining a second ultrasonic image at the adjusted pressure; and
determining whether the second ultrasonic image meets the image quality requirement, and if so, then determining the adjusted pressure as the pressure applied by the scanning assembly to the tissue to be scanned during ultrasonic diagnostic scanning.

4. The method according to claim 3, wherein said determining whether the second ultrasonic image meets the image quality requirement comprises: determining, on the basis of a trained deep learning network, whether the second ultrasonic image meets the image quality requirement.

5. The method according to claim 3, wherein the step of said determining, within the pressure range, a pressure applied by the scanning assembly to the tissue to be scanned during ultrasonic diagnostic scanning comprises:
if the adjusted pressure reaches the minimum value of the pressure range, then controlling the scanning assembly to stop reducing the pressure applied on the tissue to be scanned; and
if the adjusted pressure reaches the maximum value of the pressure range, then controlling the scanning assembly to stop increasing the pressure applied on the tissue to be scanned.

6. The method according to claim 4, further comprising: if the second ultrasonic image does not meet the image quality requirement or if the adjusted pressure reaches the maximum value or the minimum value of the pressure range, then issuing a corresponding indication signal.

7. The method according to claim 1, wherein before the controlling a scanning assembly to apply an initial pressure to a tissue to be scanned of an subject, the method further comprises: determining, on the basis of received basic information of the subject, a maximum pressure applied by the scanning assembly to the tissue to be scanned, wherein the maximum value of the initial pressure is less than or equal to the maximum pressure.

8. The method according to claim 7, wherein the method further comprises: receiving a feedback from a pressure sensor indicating whether the value of the pressure applied by the scanning assembly to the tissue to be scanned exceeds the maximum pressure, and if a determination result is “yes,” then controlling the scanning assembly to release the initial pressure.

9. The method according to claim 1, wherein the method further comprises:
receiving an emergency stop signal based on an operation of the subject so as to stop the ultrasonic diagnostic scanning.

10. The method according to claim 9, wherein the scanning assembly comprises an ultrasonic transducer, and the method further comprises: controlling, on the basis of the emergency stop signal, the ultrasonic transducer to withdraw from a current position.

11. An ultrasonic scanning control device, comprising:
a control module, configured to control a scanning assembly to apply an initial pressure to a tissue to be scanned of an subject;
a first image obtaining module, configured to obtain first ultrasonic images at different initial pressures;
a pressure range determination module, configured to determine a pressure range on the basis of the first ultrasonic images obtained at the different initial pressures; and
a pressure determination module, configured to determine, within the pressure range, a pressure applied by the scanning assembly to the tissue to be scanned during ultrasonic diagnostic scanning;
wherein the control module is further configured to perform ultrasonic diagnostic scanning on the tissue at the determined pressure within the pressure range.

12. The device according to claim 11, wherein the pressure range determination module comprises:
a first image quality determination unit, configured to determine, on the basis of a trained deep learning network, whether the first ultrasonic images meet an image quality requirement; and
a pressure range determination unit, configured to determine the pressure range on the basis of initial pressures corresponding to the first ultrasonic images meeting the image quality requirement.

13. The device according to claim 11, wherein the pressure determination module comprises:
a pressure adjustment unit, configured to receive a pressure adjustment signal based on an operation of the subject so as to adjust the current initial pressure and send the adjusted initial pressure to the control module, wherein the control module is configured to control the scanning assembly to apply the adjusted pressure to the tissue to be scanned of the subject;
a second image obtaining unit, configured to obtain a second ultrasonic image at the adjusted pressure;
a second image quality determination unit, configured to determine whether the second ultrasonic image meets an image quality requirement; and
a pressure determination unit, wherein if the second ultrasonic image meets the image quality requirement, then the pressure adjustment unit determines the adjusted pressure as the pressure applied by the scanning assembly to the tissue to be scanned during ultrasonic diagnostic scanning.

14. The device according to claim 13, wherein the second image quality determination unit is configured to determine, on the basis of a trained deep learning network, whether the second ultrasonic image meets the image quality requirement.

15. The device according to claim 11, further comprising a maximum pressure determination module, the maximum pressure determination module being configured to determine, on the basis of inputted subject information, the maximum pressure applied by the scanning assembly to the tissue to be scanned, wherein the maximum value of the initial pressure is less than or equal to the maximum pressure.

16. The device according to claim 11, wherein the scanning assembly comprises an ultrasonic transducer, and the control module is further configured to:
receive an emergency stop signal based on an operation of the subject so as to stop the ultrasonic scanning, and control the ultrasonic transducer to withdraw from a current position.

17. (canceled)

18. An ultrasonic imaging system, comprising:
a scanning assembly, configured to perform reference scanning and formal scanning on a tissue to be scanned of an subject so as to respectively obtain a reference image and an ultrasonic diagnostic image; and
a controller, the controller being configured to perform the following operations:
controlling, in the reference scanning, the scanning assembly to apply a gradually increasing initial pressure to the tissue to be scanned;
determining a pressure range on the basis of reference images obtained at different initial pressures; and
adjusting the initial pressure within the pressure range on the basis of a pressure adjustment signal sent remotely, and using the same as a pressure applied by the scanning assembly to the tissue to be scanned during the formal scanning; and
performing ultrasonic diagnostic scanning on the tissue at the determined pressure within the pressure range.

19. The system according to claim 18, further comprising a pressure sensor, wherein the pressure sensor is configured to sense the pressure applied by the scanning assembly to the tissue to be scanned and send the same to the controller, and the controller is further configured to:
determine, on the basis of basic information of the subject, the maximum pressure that is acceptable to be applied to the tissue to be scanned, determine whether the pressure sent by the pressure sensor exceeds the maximum pressure, and if so, control the scanning assembly to release the initial pressure from the tissue to be scanned.

20. The system according to claim 18, wherein the controller is further configured to:
determine whether a reference image obtained at the adjusted initial pressure meets an image quality requirement, and if the reference image obtained at the adjusted initial pressure meets the image quality requirement, determine the adjusted initial pressure as a pressure applied by the pressing scanning assembly to the tissue to be scanned during the formal scanning.
